(19) United States
(12) Patent Application Publication
(10) Pub. No.: US 2022/0080088 A1
(43) Pub. Date: Mar. 17, 2022

(54) CONNECTOR WITH FUNCTION OF SPOUT OPENER

(71) Applicant: CIMILRE CO., LTD., Cheonan-si (KR)

(72) Inventor: Sang Ha KIM, Seoul (KR)

(73) Assignee: CIMILRE CO., LTD., Cheonan-si (KR)

(21) Appl. No.: 17/105,443

(22) Filed: Nov. 25, 2020

(30) Foreign Application Priority Data
Sep. 11, 2020 (KR) 10-2020-0116673

(51) Int. Cl. A61M 1/06

(52) U.S. Cl. CPC A61M 1/062

(57) ABSTRACT

The present invention relates to a connector with a function of a spout opener, which is adapted to connect a breast milk suction member for introducing breast milk thereinto to a breast milk storage pack for storing the breast milk therein, the connector including: a body with a hollow portion formed therein; and a connection part located on the body in such a manner as to couple at least one of the breast milk suction member, a spout of the breast milk storage pack, and a spout cap for opening and closing the spout thereto.

### CROSS REFERENCE TO RELATED APPLICATION OF THE INVENTION

The present application claims the benefit of Korean Patent Application No. 10-2020-0116673 filed in the Korean Intellectual Property Office on Sep. 11, 2020, the entire contents of which are incorporated herein by reference.

### BACKGROUND OF THE INVENTION

### Field of the Invention

The present invention relates to a connector with the function of a spout opener, and more particularly, to a connector that is capable of connecting a breast milk suction member and a spout of a breast milk storage pack to each other and easily opening a spout cap.

### Background of the Related Art

If it is desired that a mother's breast milk is fed to her baby, generally, she breastfeeds her baby or collects her breast milk expressed through a breast pump to a general bottle to feed the breast milk to her baby.

The breast milk remaining in the bottle after fed to the baby is left at a room temperature for given hours so as to be fed again to the baby, and as time passes, however, the breast milk is slowly oxidized and rotten in the bottle and is thus deteriorated badly so that it cannot be fed to the baby again.

Accordingly, the bottle in which a given amount of the breast milk remains is stored in a freezer, and so as to feed the breast milk to the baby again, in this case, the breast milk has to be heated up to a given temperature (about)40° appropriate for the baby, which causes many inconveniences. In particular, if the breast milk is defrosted by means of a microwave oven or it is boiled in hot water, the nutrients in the breast milk may be destroyed, thereby making it impossible to supply the nutrients necessary to the baby.

When the mother and the baby are outside their house, that is, go on a picnic, also, it is hard that the bottle is stored in the freezer, and if the expressed breast milk is collected to the bottle and is stored for a long period of time because the mother is in a different place from her baby to thus make it impossible to breastfeed her baby, the breast milk may be contaminated so that the contaminated breast milk may be fed to the baby to cause the baby's health to become bad.

So as to avoid the above-mentioned difficulties, if the breast milk remaining in the bottle after fed is thrown away, it may become an unnecessary waste.

So as to solve such problems, accordingly, the breast milk collected to the bottle through the breast pump moves to a breast milk storage pack, and the breast milk storage pack is sealed to prevent the contact with air, so that the breast milk in the beast milk storage pack is kept safe, without being rotten. Further, the breast milk storage pack has scales indicated thereon to allow an appropriate amount of breast milk to be poured into the bottle.

While the collected breast milk to the bottle through the breast pump is being moved to the breast milk storage pack, however, the breast milk may be lost.

So as to solve the loss of the breast milk, the same applicant as this invention has invented a connector that connects a breast milk suction member located on a breast pump to express breast milk to a spout of a breast milk storage pack.

However, a spout cap for opening and closing the spout is small-sized and is also firmly fastened to the spout, and as a result, actually, it is hard for a mother who has weak wrists and strength through her delivery to easily open the spout cap.

### SUMMARY OF THE INVENTION

Accordingly, the present invention has been made in view of the above-mentioned problems occurring in the related art, and it is an object of the present invention to provide a connector that is capable of connecting a breast milk suction member and a spout of a breast milk storage pack to each other and easily opening a spout cap.

To accomplish the above-mentioned object, according to the present invention, there is provided a connector with the function of a spout opener, which is adapted to connect a breast milk suction member for introducing breast milk thereinto to a breast milk storage pack for storing the breast milk therein, the connector including: a body with a hollow portion formed therein; and a connection part located on the body in such a manner as to couple at least one of the breast milk suction member, a spout of the breast milk storage pack, and a spout cap for opening and closing the spout thereto.

According to the present invention, desirably, the connection part includes a breast milk suction member fastening part located on one side of the body in such a manner as to be coupled to and separated from the breast milk suction member.

According to the present invention, desirably, the connection part includes a spout fastening part located on the other side of the body in such a manner as to be coupled to and separated from the spout.

According to the present invention, desirably, the connection part includes a cap fastening part located on the other side of the body in such a manner as to be coupled to and separated from the spout cap.

According to the present invention, desirably, the cap fastening part has locking grooves formed along an inner peripheral surface thereof in such a manner as to be locked onto grasping protrusions formed on an outer peripheral surface of the spout cap.

### BRIEF DESCRIPTION OF THE DRAWINGS

The above and other objects, features and advantages of the present invention will be apparent from the following detailed description of the preferred embodiments of the invention in conjunction with the accompanying drawings, in which:

FIGS. 1 to 5 are perspective, plan, bottom, front, and sectional views showing a connector with the function of a spout opener according to a first embodiment of the present invention;

FIG. 6 is a sectional view showing a state where a breast milk suction member and a spout are coupled to the connector with the function of a spout opener of FIG. 5;

FIGS. 7 and 8 are perspective and sectional views showing a state where a spout cap is coupled to the connector with the function of a spout opener of FIG. 5;

FIG. 9 illustrates front and bottom views showing other examples of a body of FIG. 1; and

FIGS. 10 and 11 are front and sectional views showing a connector with the function of a spout opener according to a second embodiment of the present invention.

### DETAILED DESCRIPTION OF THE EMBODIMENTS

Hereinafter, embodiments of the present invention will be in detail described so that they may be carried out easily by those having ordinary skill in the art.

In applying reference numerals to the components of the drawings, it should be noted that the corresponding parts in the embodiments of the present invention are indicated by corresponding reference numerals even if they appear on different figures.

Further, if it is determined that the detailed explanation on the well known technology related to the present invention makes the scope of the present invention not clear, the explanation will be avoided for the brevity of the description.

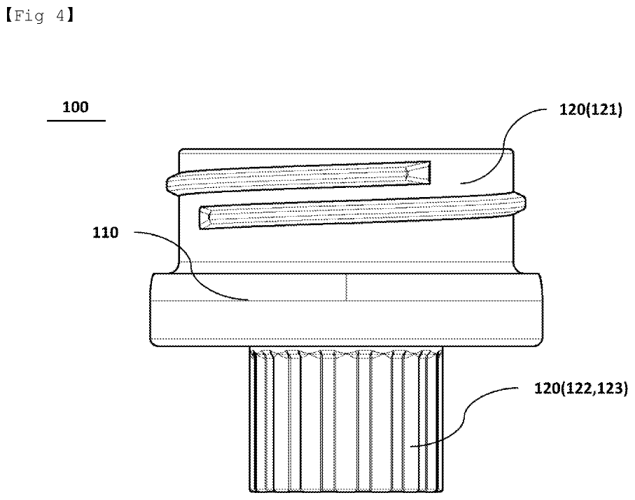

Before the detailed description of the present invention, an “upward” direction is toward a breast milk suction member fastening part 121 with respect to a spout fastening part 122 as shown in FIG. 4, and a “downward” direction is toward the spout fastening part 122 with respect to the breast milk suction member fastening part 121.

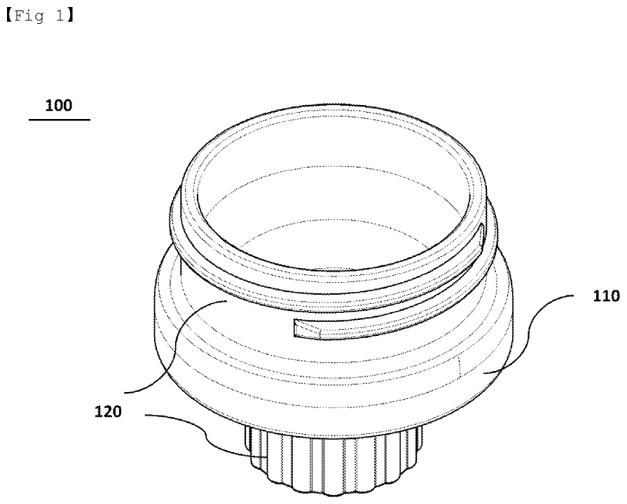

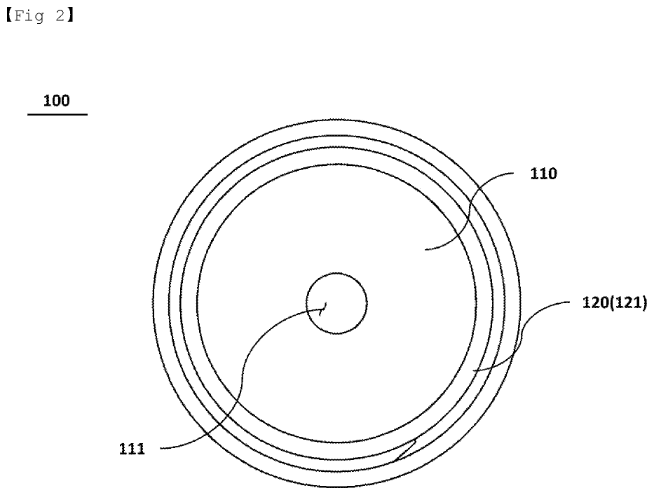

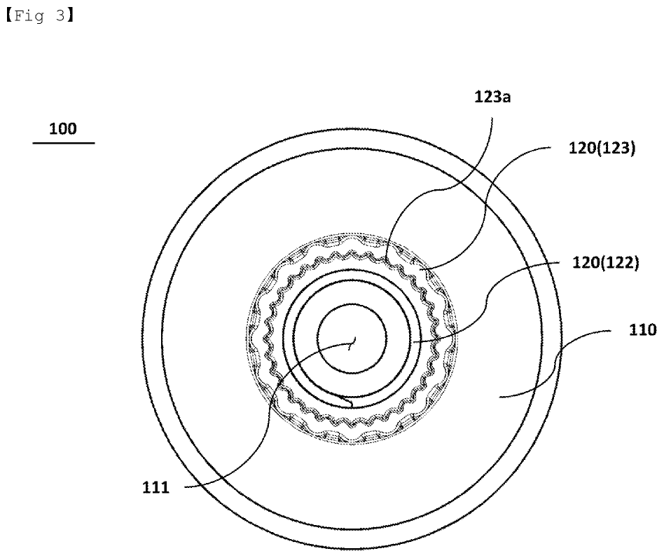

Hereinafter, an explanation on a connector with the function of a spout opener according to a first embodiment of the present invention will be in detail given with reference to FIGS. 1 to 9B.

FIGS. 1 to 5 are perspective, plan, bottom, front, and sectional views showing a connector with the function of a spout opener according to a first embodiment of the present invention.

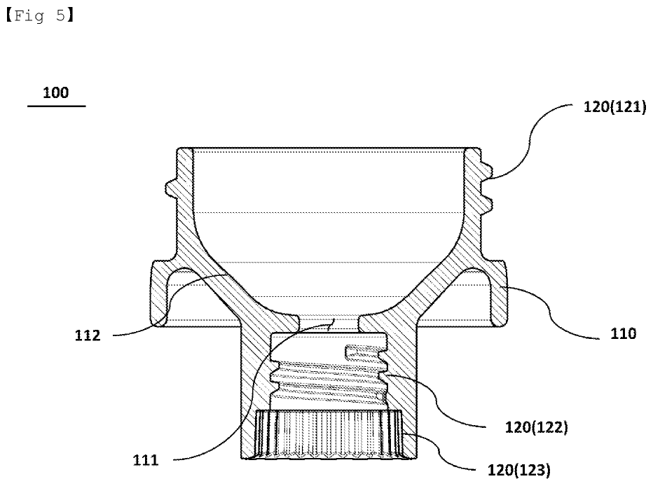

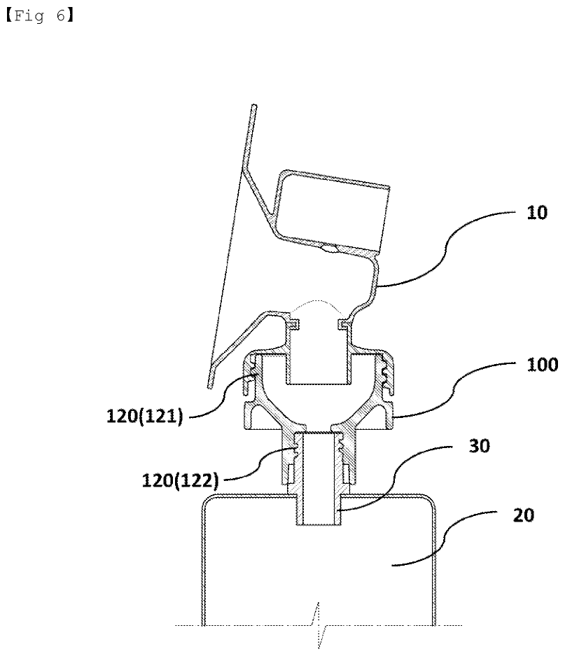

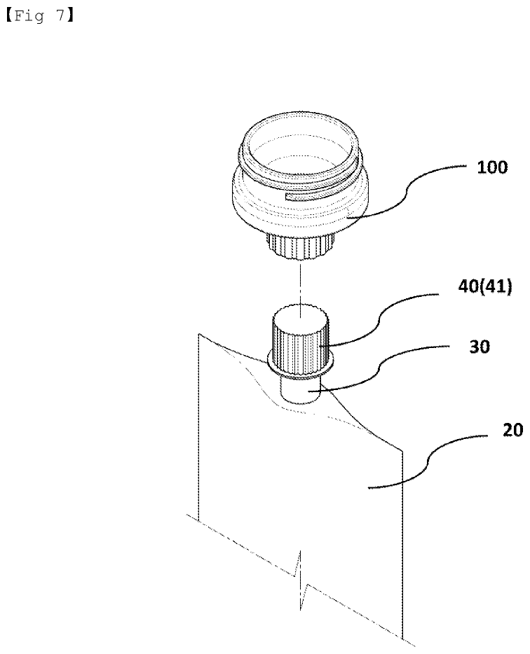

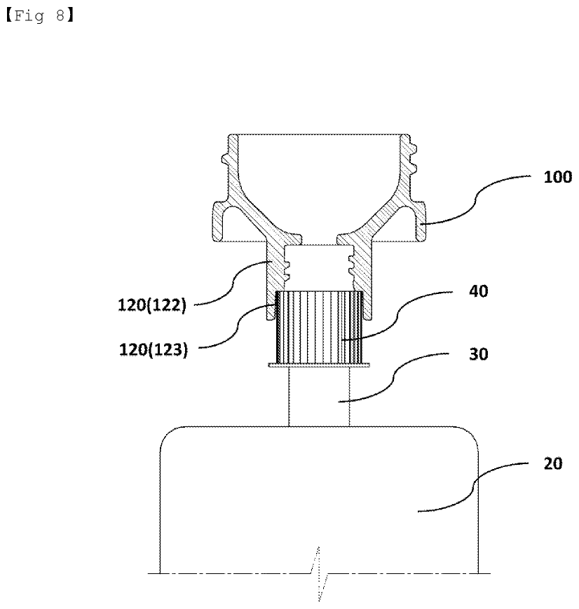

FIG. 6 is a sectional view showing a state where a breast milk suction member and a spout are coupled to the connector with the function of a spout opener of FIG. 5, and FIGS. 7 and 8 are perspective and sectional views showing a state where a spout cap is coupled to the connector with the function of a spout opener of FIG. 5.

Referring first to FIGS. 1 to 8, a connector 100 with the function of a spout opener (hereinafter referred to as ‘connector’) connects a breast milk suction member 10 adapted to introduce breast milk thereinto to a breast milk storage pack 20 adapted to store the breast milk therein.

The connector 100 includes a body 110 with a hollow portion 111 formed therein and a connection part 120 located on the body 110 in such a manner as to communicate with the hollow portion 111 and adapted to couple at least one of the breast milk suction member 10, a spout 30 of the breast milk storage pack 20, and a spout cap 40 for opening and closing the spout 30 thereto.

In more detail, the connector 100 is made of a known material with given stiffness like plastic, steel, and so on, and the connection part 120 has a plurality of fastening parts adapted to fasten the breast milk suction member 10, the spout 30, and the spout cap 40 thereto.

Accordingly, the connection part 120 includes the breast milk suction member fastening part 121 located on one side of the body 110, communicating with the hollow portion 111, and coupled to and separated from the breast milk suction member 10 and a spout fastening part 122 located on the other side of the body 110, communicating with the hollow portion 111, and coupled to and separated from the spout 30.

As shown, the spout that is generally distributed in the market has a circular end periphery and a screw thread formed therealong, so that it can be easily coupled to and separated from the spout cap.

Also, the breast milk suction member 10 as invented by the same applicant as this invention has a coupling way through a screw thread formed thereon, and accordingly, the connector 100 has the shape of a cylinder in such a manner as to have a screw thread formed on an outer peripheral surface of the breast milk suction member fastening part 121 and a screw thread formed on an inner peripheral surface of the spout fastening part 122.

The breast milk suction member fastening part 121, the body 110, and the spout fastening part 122 communicate with one another to sequentially move the breast milk introduced into the breast milk suction member 10 therealong.

The body 110 has a slant surface 112 formed along the inner periphery thereof in such a manner as to allow the hollow portion 111 to become reduced in diameter from the breast milk suction member fastening part 121 toward the spout fastening part 122, and accordingly, the inside of the body 110 has the shape of a cone.

As the inside of the body 110 has the conical shape, in detail, the breast milk can be prevented from stackingly remaining in an interior of the body 110.

The connection part 120 further includes a cap fastening part 123 located on the other side of the body 110 in such a manner as to be coupled to and separated from the spout cap 40.

In more detail, one side end periphery of the spout fastening part 122 is connected to the body 110 in such a manner as to communicate with the hollow portion 111, and one side end periphery of the cap fastening part 123 is connected to the other side end periphery of the spout fastening part 122 in such a manner as to communicate with the spout fastening part 122.

The cap fastening part 123 has locking grooves 123a locked onto grasping protrusions 41 formed on an outer peripheral surface of the spout cap 40.

As shown in FIGS. 7 and 8, the grasping protrusions 41 are spaced apart from one another by a given distance on the outer peripheral surface of the spout cap 40 in a longitudinal direction of the spout 30.

The locking grooves 123a are spaced apart from one another by a given distance along an inner peripheral surface of the cap fastening part 123 in the longitudinal direction of the spout 30 in such a manner as to be locked onto the grasping protrusions 41.

In a state where the spout cap 40 is fitted to the cap fastening part 123, in detail, the body 110 or the connection part 120, which has a relatively larger size than the spout cap 40 so that it can be easily grasped, rotates to allow a mother who has weak wrists to open the spout cap 40 easily.

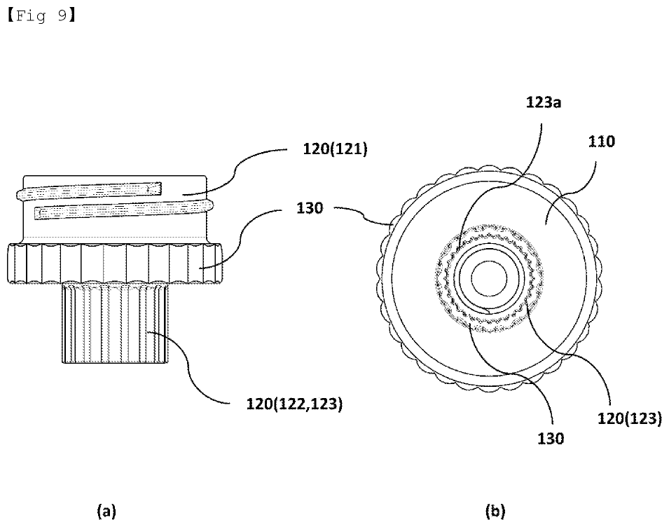

FIG. 9 illustrates front and bottom views showing other examples of a body of FIG. 1.

Referring to FIGS. 9A and 9B, the connector 100 has a plurality of friction protrusions 130 spaced apart from one another by a given distance along the outer peripheral surface of the body 110 in such a manner as to allow a user inclusive of a mother to grasp the connector 100 firmly.

Further, the friction protrusions 130 can be spaced apart from one another by a given distance along the outer peripheral surface of the spout fastening part 122.

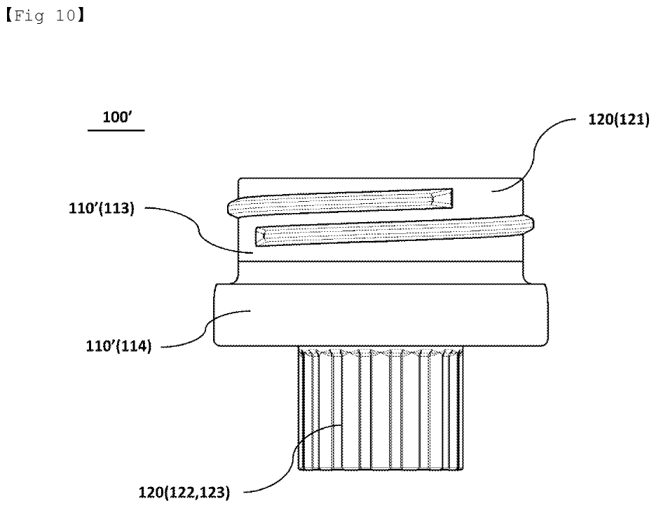

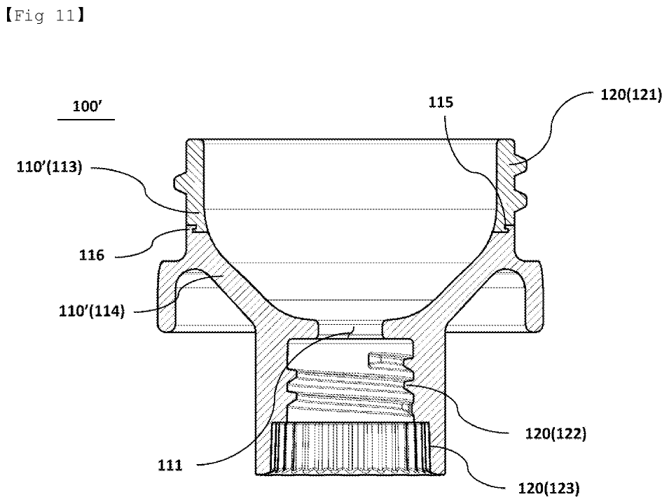

Hereinafter, an explanation on a connector with the function of a spout opener according to a second embodiment of the present invention will be in detail given with reference to FIGS. 10 and 11, and for the brevity of the description, the parts repeated in the first embodiment of the present invention will be not explained.

FIGS. 10 and 11 are front and sectional views showing a connector with the function of a spout opener according to a second embodiment of the present invention.

After the breast milk suction member 10 and the spout 30 are coupled to the connector 100 suggested in the first embodiment of the present invention, they are fixed to the connector 100 and are thus restricted in rotation.

In the state where the spout 30 is completely coupled to the connector 100, if an edge of the breast milk storage pack 20 comes into contact with the mother's breast, the mother feels uncomfortable, so that the breast milk storage pack has to be changed in position. In this case, the coupling state of the spout 30 becomes loose to cause the spout 30 to be separated from the connector 100, or otherwise, the breast milk may leak to the outside.

If the breast milk storage pack 20 rotates by means of unexpected external forces, further, the breast milk suction member 10 may be separated from the mother's breast to stop expressing the breast milk.

So as to solve such problems, accordingly, a connector 100′ according to a second embodiment of the present invention includes a body 110′ having a first body 113 adapted to locate the breast milk suction member fastening part 121 on one side thereof and a second body 114 adapted to allow the other side of the first body 113 to be rotatably located on one side thereof and to locate the spout fastening part 122 on the other side thereof.

In detail, the body 110′ is divided into the first body 113 and the second body 114 to allow the restriction in rotation of the breast milk storage pack 20 to be released, so that the breast milk suction member 10 does not have any influence on the rotation of the breast milk storage pack 20.

In more detail, the connector 100′ has a coupling groove 115 formed along an outer peripheral surface of the first body 113 and a locking protrusion 116 formed along an inner peripheral surface of the second body 114 in such a manner as to be inserted into the coupling groove 115.

Through the coupling structure between the coupling groove 115 and the coupling protrusion 116, accordingly, the first body 113 and the second body 114 do not have any influence on each other in rotation.

As described above, the connector according to the embodiments of the present invention can connect the breast milk suction member and the spout to each other through the plurality of fastening parts.

Further, the connector according to the embodiments of the present invention is provided with the cap fastening part to which the spout cap is fastened, so that the body, which has a relatively larger size than the spout cap so that it can be easily grasped, rotates to easily open the spout cap.

While the present invention has been described with reference to the particular illustrative embodiments, it is not to be restricted by the embodiments but only by the appended claims. It is to be appreciated that those skilled in the art can change or modify the embodiments without departing from the scope and spirit of the present invention. Therefore, it should be understood that the invention covers all the modifications, equivalents, and replacements within the idea and technical scope of the invention.

What is claimed is:

1. A connector with the function of a spout opener, which is adapted to connect a breast milk suction member for introducing breast milk thereinto to a breast milk storage pack for storing the breast milk therein, the connector comprising:
a body with a hollow portion formed therein; and
a connection part located on the body in such a manner as to couple at least one of the breast milk suction member, a spout of the breast milk storage pack, and a spout cap for opening and closing the spout thereto.

2. The connector according to claim 1, wherein the connection part comprises a breast milk suction member fastening part located on one side of the body in such a manner as to be coupled to and separated from the breast milk suction member.

3. The connector according to claim 1, wherein the connection part comprises a spout fastening part located on the other side of the body in such a manner as to be coupled to and separated from the spout.

4. The connector according to claim 1, wherein the connection part comprises a cap fastening part located on the other side of the body in such a manner as to be coupled to and separated from the spout cap.

5. The connector according to claim 4, wherein the cap fastening part has locking grooves formed along an inner peripheral surface thereof in such a manner as to be locked onto grasping protrusions formed on an outer peripheral surface of the spout cap.
